(19) United States
(12) Patent Application Publication
(10) Pub. No.: US 2002/0119178 A1
(43) Pub. Date: Aug. 29, 2002

(54) Drug eluting device for treating vascular diseases

(76) Inventors: Luc Levesque, Boucherville (CA); Marcus F. Lawrence, Chambly (CA); Bernard Bourguignon, Montreal (CA); Guy Leclerc, Rosemere (CA)

Correspondence Address: NIXON PEABODY LLP, ATTENTION: DAVID RESNICK, 101 FEDERAL STREET, BOSTON, MA, 02110, US

(21) Appl. No.: 10/080,499

(22) Filed: Feb. 22, 2002

Related U.S. Application Data
(60) Provisional application No. 60/270,605, filed on Feb. 23, 2001.

(51) Int. Cl. B05D 3/00; A61F 2/00

(52) U.S. Cl. 424/423; 427/2.28

(57) ABSTRACT

The present invention relates to a device and method for delivering locally therapeutic agents within adjacent tissues such as an arterial wall for treating vascular diseases. The device comprises i) an endovascular device, ii) an hydrophobic linker molecule containing a diazonium moiety electrodeposited onto the surface of the endovascular device, and iii) a lipophilic drug passively deposited on the linker molecule, said drug binding to the linker molecule through hydrophobic interactions for elution from the endovascular device over time. The present invention also relates to a method for preparing such device.

### BACKGROUND OF THE INVENTION

(a) Field of the Invention

The present invention relates to a device and method for delivering locally therapeutic agents within adjacent tissues such as an arterial wall for treating vascular diseases.

(b) Description of Prior Art

Although coronary angioplasty procedures reduce anginal symptoms, a high incidence of restenosis (30 to 40% within 6 months) is the “Achilles' heel” of interventional cardiology. With over one million coronary procedures performed annually around the world, the economic effect of restenosis is substantial. Systemic pharmacological approaches to prevent restenosis have failed to be effective and only coronary stenting procedure reduced restenosis rates (STRESS and BENESTENT trials). Stent deployment, however, frequently induces a new coronary occlusion known as in-stent restenosis. About 20% of stented patients develop in-stent restenosis.

Drug delivery stents, which attempted to deliver pharmacological agents to the arterial wall in the region where angioplasty was performed, have previously been reported. One of these devices, disclosed in U.S. Pat. No. 6,071,305, consists of a stent that has an interior cavity containing a therapeutic agent for sustained directional delivery directed toward an arterial lumen.

Other devices, disclosed in U.S. Pat. Nos. 5,429,634, 5,500,013 and 5,443,458 for example, are biodegradable stents, which are impregnated with therapeutic agents.

Another example of delivery devices, disclosed in U.S. Pat. No. 5,342,348, are stents that contain therapeutic agents impregnated with a matrix of filaments, which may be woven or laminated onto the stent.

Still another example of delivery devices, disclosed in U.S. Pat. Nos. 5,649,977 and 5,700,286, includes stents, which are coated with a polymer capable of absorbing and releasing therapeutic drugs.

Another example, described in U.S. Pat. No. 5,972,027, consists of a stent manufactured from powdered metal or polymers with a specific porosity. Therapeutic drugs can then be compressed into the pores of the stent to be locally released.

U.S. Pat. No. 5,234,456 discloses a hydrophilic stent, which can include a therapeutic agent disposed within the hydrophilic material of the stent.

Therefore, it would be highly desirable to be provided with a drug delivery system that would take advantage of lipophilic properties of therapeutic agents to retain them onto the stent for sustained-release thereafter.

It would also be highly desirable to be provided with a new method for loading an endovascular device with a drug for sustained-release.

### SUMMARY OF THE INVENTION

One object of the present invention is to provide a deposition process of pharmacological therapeutic agents on the surface of an angioplastic device for preventing restenosis post-angioplasty or on other medical devices dedicated for treatment of vascular diseases.

Another object of the present invention is to provide a new endovascular device for local and sustained delivery of pharmacological therapeutic agents into the arterial wall for treating vascular diseases or for preventing restenosis post-angioplasty.

In accordance with the present invention there is provided a method to functionnalize an endovascular device for molecule coating. The endovascular device may be functionalized with molecules containing a diazonium (NΞON) moiety. The functionalized surface of the endovascular device will then bind therapeutic molecules and retain these agents for subsequent release into a target tissue. In accordance with the present invention, there is provided a method for loading a drug onto an endovascular device, said method comprising the steps of :

electrodepositing an hydrophobic molecule containing a diazonium moiety onto the surface of an endovascular device to obtain a functionalized surface of said device; and

depositing passively a lipophilic drug onto said functionalized surface, said drug binding to the diazonium moiety of the molecule for slow elution into a tissue when said device is brought in contact with said tissue in vivo.

Still in accordance with the present invention, this method permits to functionnalize any stainless steel endovascular device with molecules containing a diazonium moiety.

Still in accordance with the present invention, this method permits to bind any lipophilic therapeutic agent provided from any drug class on any stainless steel endovascular device.

The method of the present invention allows for obtaining a drug eluting coated device on which the therapeutic agent is effectively bound and uniformly deposited. Following deposition treatment, no adverse effects are observed in coated stents in vi tro (mechanical properties) and in vivo (clotting, thrombogenicity).

Further in accordance with the present invention, there is provided a drug-eluting endovascular device comprising:

an endovascular device;

an hydrophobic linker molecule containing a diazonium moiety electrodeposited onto the surface of the endovascular device; and

a lipophilic drug passively deposited on the linker molecule, said drug binding to the linker molecule through hydrophobic interactions for elution from the endovascular device over time.

Still in accordance with the present invention, the device will release the desired therapeutic agent over the course of time into the wall of a blood vessel or into a target tissue.

Further in accordance with the present invention, there is provided a method for preventing vascular diseases such as restenosis in a coronary and/or peripheral artery comprising implanting an endovascular device as defined above at a site of potential restenosis such as coronary and/or peripheral artery in a patient in need of such a treatment.

Therefore, the present invention takes advantage of lipophilic properties of therapeutic agents and hydrophobic moieties of a linker molecule, such as a diazonium-containing molecule, used to bind the therapeutic agents to an endovascular device such that it will blend within the cell membrane therefore, delivering directly the active molecule within the cell, increasing the efficiency of transfer.

Moreover, the present invention also takes advantage of the hydrophobic nature of the cellular membrane, which possesses an enhanced affinity for lipophilic therapeutic drugs. Therefore, the drugs are less likely to be washed out in the blood stream, which is relatively more hydrophilic in nature. As a result, this increases efficacy of transfer between the device and the adjacent arterial smooth muscle cells.

This strategy contrasts with all other methods of drug delivery since other methods do not take in account the lipophilic properties of the cell membrane. For example, biodegradable, polymer coated, porous or hydrophilic-coated stents will release the drugs not only within the cell membrane, but also in the blood stream since there is no common denominator between the therapeutic agent and the cell membrane.

By the term functionalization, it is intended to mean the application of a reagent, such as a diazonium moiety, to a solid surface that will permit molecule coating.

By the term endovascular device, it is intended to mean any device used endovascularly such as for angioplasty or for treating aneurysms. Such device may be without limitation a stent, or a wire or any other device to which a person of the art may think of for the treatment of vascular diseases such as prevention of an uncontrolled proliferative lesion or the treatment of an aneurysm. The term endovascular device is also meant to include any prosthesis to be implanted within a vessel or within other body conduit such as, but not restricted to, the bile duct or urethra for the purpose of endovascular treatment.

### BRIEF DESCRIPTION OF THE DRAWINGS

Having thus generally described the nature of the invention, reference will now be made to the accompanying drawings, showing by way of illustration a preferred embodiment thereof, and wherein:

FIG. 1 illustrates a schematic electrodeposition set-up used for diazonium functionnalization of a stainless steel surface for passive deposition of a lypophilic drug;

FIG. 2 represents examples of molecules containing a diazonium moiety that can be electrodeposited onto a stainless steel endovascular device;

FIG. 3 is a schematic illustration of a stent coated with a drug in accordance with one embodiment of the present invention.

FIG. 4 is a schematic cross-section view taken along lines III-III on FIG. 3. illustrating a drug delivery stent according to one embodiment of the present invention positioned in an arterial lumen;

FIG. 5 is a bar graph demonstrating the advantage of functionnalization of stainless steel 316L discs with 4-bromobenzenediazonium to retain tritiated actinomycin D loaded onto the discs with either acetonitrile or ethanol;

FIG. 6 is a bar graph illustrating the capacity of functionalized stainless steel discs in accordance with the present invention to load and retain tritiated actinomycin D, loaded with either water, acetonitrile or ethanol, immediately following a wash in water (after drug loading) or following a 10-day elution in a physiological solution;

FIG. 7 is a bar graph illustrating the effect of various concentrations of 4-bromobenzenediazonium upon loading of tritiated actinomycin D and following 8 days of elution in a physiologic medium;

FIG. 8 illustrates a bar graph representing loading and retention of tritiated actinomycin D onto stainless steel discs functionalized in accordance with the present invention with various molecules containing a diazonium moiety;

FIG. 9 is graph illustrating dose-response curves of anti-proliferative therapeutic drugs, on the inhibition of vascular muscle cell proliferation; and

FIGS. 10A to 10G are bar graphs representing the effect of the elution of bromobenzenediazonium alone (FIG. 10A), or non-functionalized discs loaded with actinomycin D (FIG. 10B), or functionalized discs loaded with actinomycin D (FIG. 10C), rapamycin (FIG. 10D), paclitaxel (FIG. 10E), doxorubicin (FIG. 10F), and colchicine (FIG. 10G) on cell proliferation.

### DETAILED DESCRIPTION OF THE INVENTION

In accordance with the present invention, there is provided a method for depositing lipophilic therapeutic agents onto an endovascular device. Therapeutic agents loaded onto the therapeutic device in accordance with the present invention are eluted over time into the adjacent arterial tissue thus preventing restenosis, thrombosis, and inflammation, to promote healing and/or to provide numerous other treatments for a period of time longer than if the therapeutic agents would have been administered alone.

The invention also relates to an endovascular device onto which hydrophobic linker molecules containing a diazonium moiety are electrodeposited to create a drug-eluting device. Therapeutic agents may then be absorbed onto the hydrophobic linker molecules, to be released over a period of time to treat vascular diseases or to reduce or eliminate restenosis in the blood vessel.

Preferred therapeutic drugs which may be delivered by the present invention belong to the following subgroups: anti-proliferative agents to prevent uncontrolled cellular proliferation and tissue growth, anti-inflammatory agents to prevent inflammation, anti-thrombotic drugs to prevent or control formation of thrombus or thrombolytics, conversion enzyme inhibitors, and other bioactive agents which regulate uncontrolled cellular proliferation, tissue growth or promotes healing of the tissue. Examples of therapeutic compounds which can be used in the present invention include, but are not limited to anti-neoplastic drugs which are subdivided in the following subclasses: alkylating agents (ex., cisplatin, melphalan), antimetabolites (ex., methotraxate, 5-fluorouracil), antibiotics (ex., actinomycin D, bleomycin, rapamycin), mitotic inhibitors (ex., vincristine, vinblastine, paclitaxel, colchicine), hormones (ex., prednisone, tamoxifen). Other drugs can be used such as anti-coagulants (ex., heparin, coumarin compounds) fibrinolytic agents (ex., streptokinase, urokinase), non-sterioidal anti-inflammatory drugs (NSAIDs) (ex., ibuprofen, naproxen), steroidal anti-inflammatory drugs (ex. prednisone, dexamethasone), sodium channel blockers (for example, lidocaine, procainamide) and calcium channel blockers (for example, nifedipine and verapamil), nitric oxide donors (ex., nitroglycerin), conversion enzyme inhibitors (ex., captopril, enalapril), angiotensine receptor antagonists (ex., losartan), alpha-adrenoceptor blockers (ex., phentolamine, prazosin), genetic material containing DNA and RNA fragments, complete expression genes, anti-bodies, prostaglandins, leukotrienes, elastin, collagen, integrins, growth factors, radioisotopes and radioactive molecules.

Therapeutic agents may be administered in accordance with the present invention either alone or in combination with other therapeutic agents as a mixture of these compounds and can contain pharmaceutically acceptable carriers and/or additional inert ingredients.

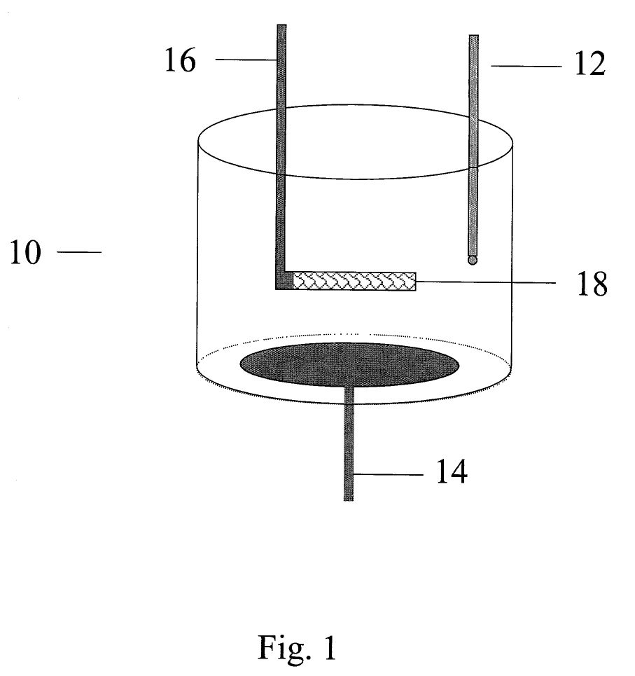

In one embodiment of the invention, the endovascular device is functionalized with a molecule containing a diazonium moiety. The functionalized surface of the endovascular device will then bind therapeutic molecules and retain these agents for subsequent release into the target tissue. FIG. 1 illustrates a schematic drawing of the electrochemical cell 10 used for aryldiazonium functionalization of stainless steel surfaces of endovascular devices such as 316L discs.

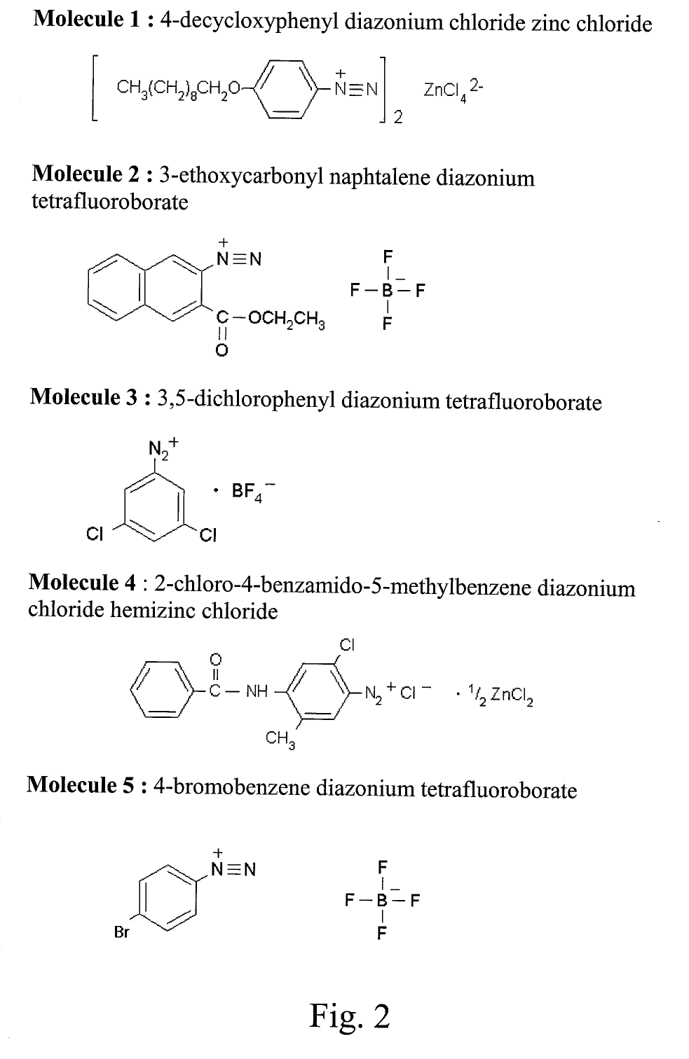

In FIG. 1, the electrochemical cell 10 is a standard three-electrode setup. A saturated Calomel electrode (SCE) was used as the reference electrode 12 and the counter electrode 14 was a circular platinum foil (3 cm²). A 316L stainless steel disk (0.8 cm² area) connected to a platinum wire 16 was used as the working electrode 18. The cell was filled with an aqueous electrodeposition solution composed of 5 mM sulfuric acid and 20 mM of an aryldiazonium-containing molecule as described in FIG. 2 for the cyclic voltammetry electrochemical process. The electrodeposition of the aryldiazonium onto the stainless steel device was applied using 2 consecutive cyclic scans ranging from −0.5 V to −1.75 V relatively to the SCE reference electrode. The current-voltage response was followed on a XY recorder. Following electrodeposition, the device was consecutively washed with water and acetonitrile to remove impurities.

As depicted in FIG. 2, several types of aryldiazonium molecules containing a diazonium moiety can be used for electrodeposition. Featured molecules are, but not limited to 4-decycloxyphenyl diazonium chloride (molecule 1), 3-ethoxycarbonyl-naphtalene-2-diazonium tetrafluoroborate (molecule 2), 3-5-dichlorophenyl diazonium tetrafluoroborate (molecule 3), 2-chloro-4-benzamido-5-methylbenzene diazonium chloride (molecule 4), and 4-bromobenzenediazonium tetrafluoroborate (molecule 5). They all have in common the diazonium moiety, which consists of two nitrogen atoms linked together by a triple bond. The chemical structure can be modified to vary the degree of retention of the therapeutic molecule onto the endovascular device.

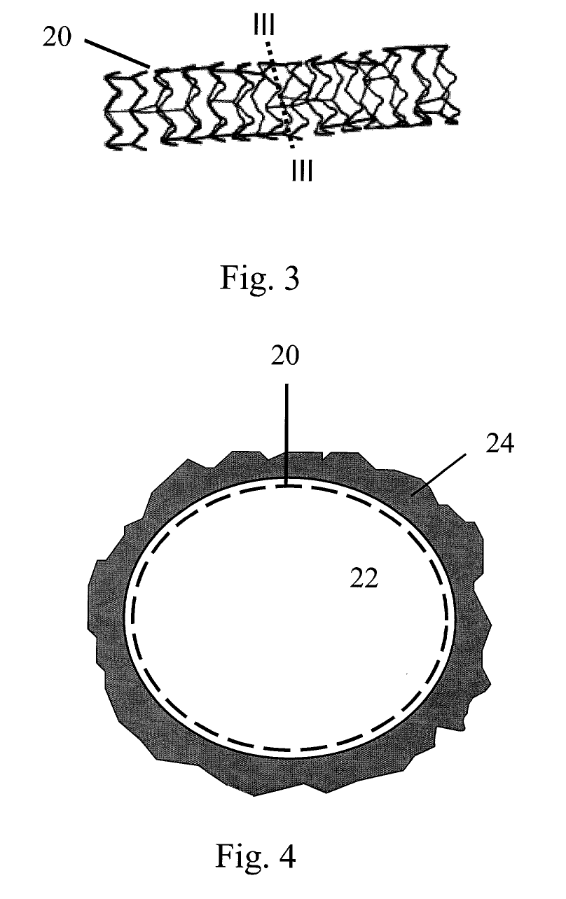

In FIG. 3, one of various aryldiazonium molecules illustrated in FIG. 2, such as bromobenzenediazonium, is electrodeposited onto the stainless steel surface of a stent 20 using the electrochemical cell depicted in FIG. 1. The electrochemical reduction of the aryldiazonium moiety involves the loss of the diazonium moiety (N₂) creating a uniform organic coating over the stainless steel stent surface. The functionalized stainless steel surface of the stent is then dipped into a volatile organic solution containing a therapeutic agent. After the stent has been dipped, it is then dried. The organic solution evaporates, creating a uniform layer of the therapeutic agent, which binds to the organic layer through hydrophobic interactions. More specifically, this organic solution may be, for example, acetonitrile or ethanol, which contains the active therapeutic agent or drug such as actinomycin D.

As seen in FIG. 4, in accordance with one embodiment of the present invention, the stainless steel stent 20 is prepared with a coating of therapeutic drug. When expanded within a body lumen 22 by any known method such as by inflation of a balloon catheter or by use of shape memory materials, the drug then elutes from the surface of the stent 20 and enters cells 24 adjacent to the stent 20.

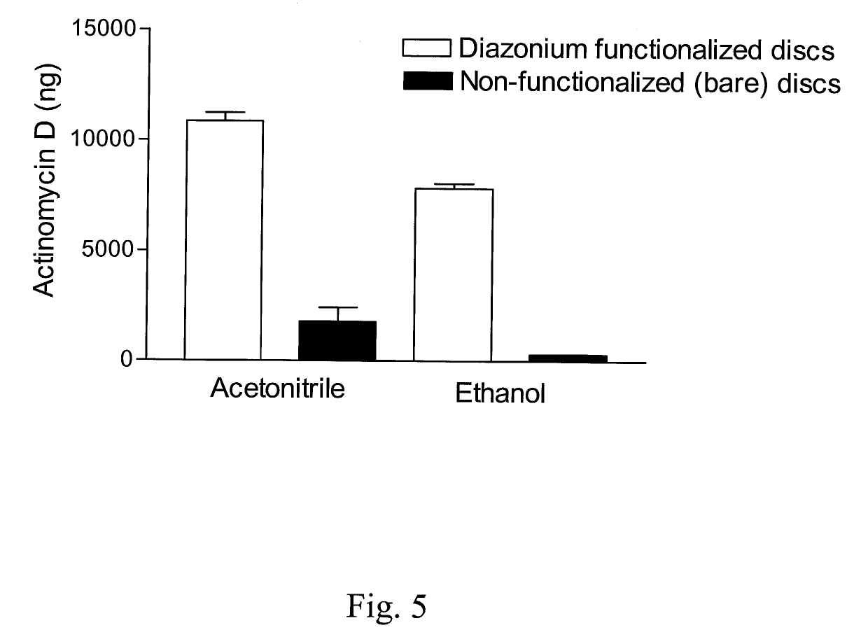

FIG. 5 illustrates the necessity of the presence of a molecule containing a diazonium moiety to retain tritiated actinomycin D deposited on the surface of stainless steel 316L discs. In this experiment, stainless steel 316L discs, which are made out of the same material as the stainless steel stents and other endovascular devices, are either functionalized with 4-bromobenzenediazonium or left bare. The discs are then exposed to a solution containing 30 μg of tritiated actinomycin D whereas the solvent is acetonitrile or ethanol. Following dipping, the discs are left to dry at room temperature until the solvent evaporates. The discs are first washed in deionized water for 5 minutes followed by a 5-minute wash in a physiologic solution. The discs are then counted in a scintillation counter.

It was observed that functionalization of 316L discs with 4-bromobenzenediazonium increases significantly retention of the tritiated actinomycin D compared to non-functionalized 316L discs in all conditions. Furthermore, acetonitrile and ethanol are both suitable to immobilize the tritiated actinomycin D.

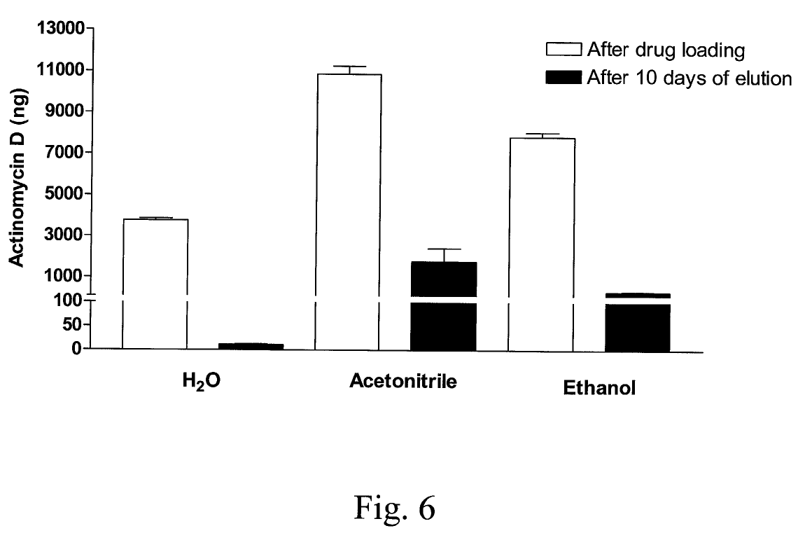

FIG. 6 illustrates the loading and retention capacity of tritiated actinomycin D immobilized as described previously onto stainless steel 316L discs, with the exception however that water was also used as solvent for immobilizing tritiated actinomycin D. Following immobilization, the discs were first washed for 5 minutes in deionized water followed by a 5-minute wash in a physiologic solution. The loading of tritiated actinomycin D onto the stainless steel discs varied according of the type of solvent used: acetonitrile>ethanol>water. Following 10 days of elution, substantial amounts of tritiated actinomycin D remained onto discs when actinomycin D was loaded with acetonitrile or ethanol. The use of an inorganic solvent such as water to load discs in accordance with the present invention provided a very low capacity to retain tritiated actinomycin D onto the stainless steel discs. This result further denotes the notion that this delivery system is based on the requirement of hydrophobic reagents such as the aryldiazonium, organic solvent and lipophilic therapeutic drugs.

After 10 days of elution, approximatively 20% of actinomycin D remained on the discs loaded with acetonitrile. These results demonstrate that in these eluting conditions, over 40 days of sustained drug release could be attained in vitro.

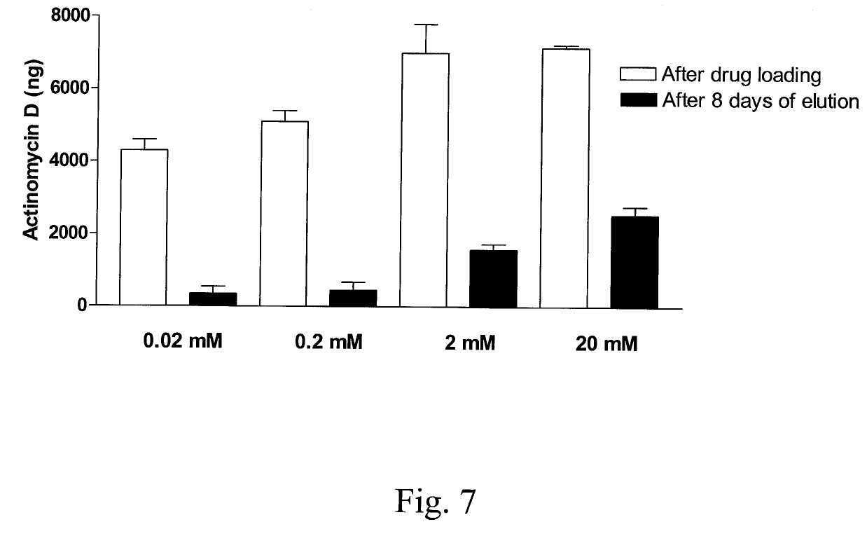

FIG. 7 illustrates the effect of varying concentrations of the 4-bromobenzenediazonium solution on the loading and retention of 30 μg of tritiated actinomycin D following 8 days of elution in a physiological medium. Stainless steel 316L discs were exposed to varying concentrations of 4-bromobenzenediazonium solution before electrodeposition with the set-up as described in FIG. 1. Actinomycin D loading in the ethanol solution increased 1.6 fold, from 4324±329 for 0.02 M to 7146±80 for 20 mM. However, the residual tritiated actinomycin D remaining on the discs following 8 days of elution was increased 7.3 fold when comparing the 0.02 mM 4-bromobenzenediazonium concentration (348±52) versus 20 mM (2539±43). Therefore, it can be stressed that although tritiated actinomycin D loading was marginally increased by high concentrations of 4-bromobenzenediazonium, the major effect of the varying concentration resides in the retention profile of the therapeutic drug.

Therefore, the rate of release of drugs can be modulated by varying the concentration of molecules containing the diazonium moiety, thereby providing a means to deliver therapeutic molecules as a function of time in a target tissue.

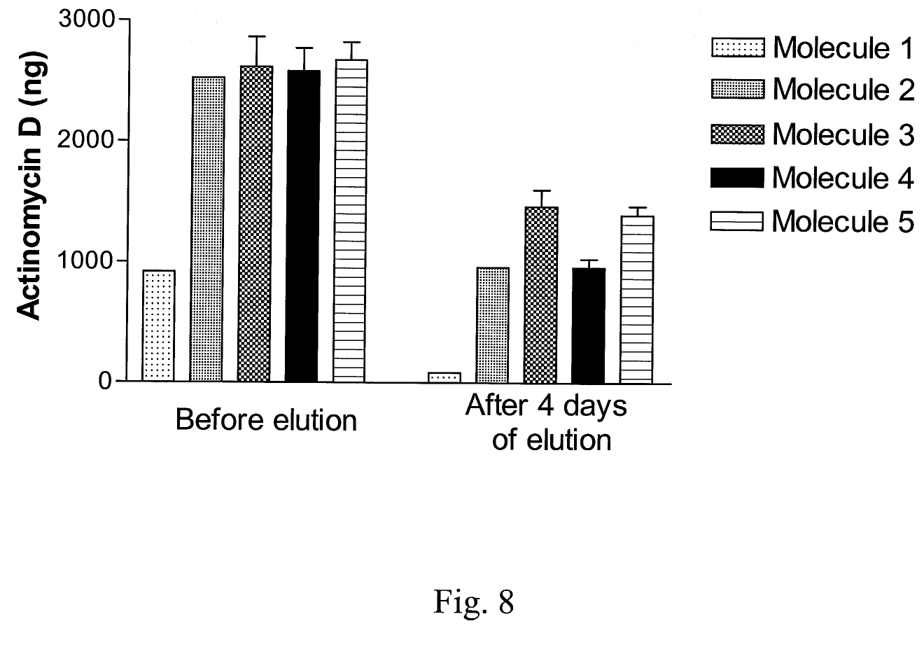

FIG. 8 illustrates the retention profiles of actinomycin D loaded onto a stainless steel disk with any one of the molecules having a diazonium moiety illustrated in FIG. 2. When 10 μg of tritiated actinomycin D was deposited onto functionalized stainless steel discs, the amount of drug retained following two 5-minute washings were similar for molecules 2, 3, 4 and 5, while retention levels was significantly lower for molecule 1. The retention capacity after 4 days of elution demonstrated that molecules 3 and 5 were the most potent to be retained onto the stainless steel surface. From these results, bromobenzenediazonium, molecule 5, was chosen for the pursuit of biology data.

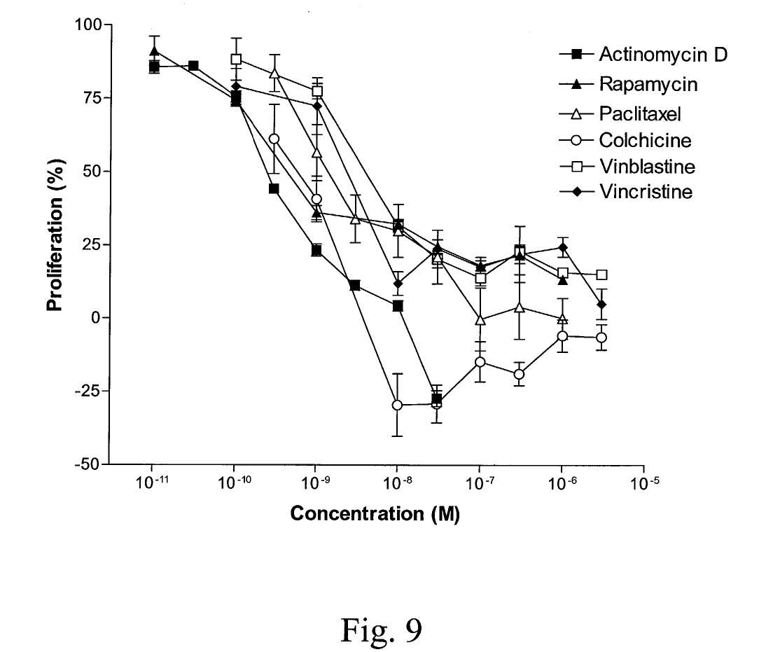

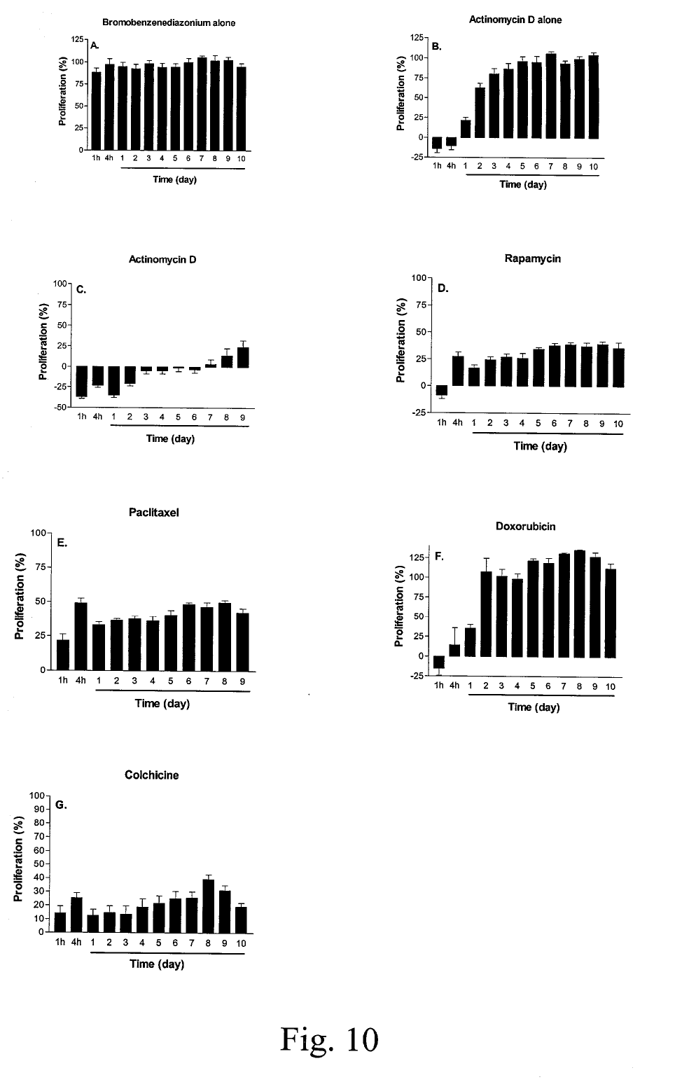

To demonstrate the possibility of loading the drug eluting device for various drugs, in vitro drug eluting experiments were performed to assess whether the sustainable release of drug could indeed inhibit cellular proliferation. A proliferation assay was performed using human saphenous vein smooth muscle cells (HSV-SMC) with cells at passage 3-5. HSV-SMC were established in 96-well plates for 24 hours then serum starved for 48 hours. Cells were cultured in culture media supplemented with 20% fetal bovine serum containing either anti-proliferative drugs at known concentrations (FIG. 9) or drugs that eluted from stainless steel discs (FIGS. 10A to 10G), and inhibition of cellular proliferation was measured. A positive control (100%) was set for cells exposed to DMEM supplemented with 20% FBS only while a negative control (0%) was set for cells exposed to only unsupplemented DMEM. Cells were stimulated for 72 hours with the anti-proliferative drug containing culture media. A solution of [3-(4,5-dimethylthiazol-2-yl)-5-(3-carboxymethoxyphenyl)-2-(4-sulfophenyl)-2H-tetrazolium](MTS, a cell proliferation marker) is then added onto the cells for 3 hours. Absorbance at 490 nm is recorded using a 96-well plate reader.

FIG. 9 illustrates the inhibition of HSV-SMC proliferation by various anti-proliferative drugs as a function of concentration. The IC₅₀ (concentration at which the proliferation is reduced by 50%) of the drugs are 8.1×10⁻¹¹ M for actinomycin D, 1.2×10⁻¹⁰ M for rapamycin, 7.4×10⁻¹⁰ M for vinblastine, 8.2×10⁻¹⁰ M for vincristine, 1.0×10⁻⁹ M for colchicine, 8.0×10⁻⁹ M for doxorubicin and 4.8×10⁻⁸ M for paclitaxel.

FIGS. 10A to 10G illustrate the effect of the elution of selected drugs illustrated in FIG. 9, from stainless steel 316L discs on HSV-SMC proliferation. In this experiment, either actinomycin D (3 μg, FIG. 10C), rapamycin (30 μg, FIG. 10D), paclitaxel (30 μg, FIG. 10E), doxorubicin (30 μg, FIG. 10F) or colchicine (30 μg, FIG. 10G) was immobilized with ethanol onto bromobenzenediazonium coated discs. Other controls were also set for actinomycin D on non-coated stainless steel discs (FIG. 10B) or bromobenzenediazonium coated discs only (FIG. 10A). The drug coated discs were placed in a conical tube containing 1 ml of DMEM supplemented with 20% FBS for 1 hour, 4 hours and then consecutive 24 hours periods of time. For each determined period of time, the culture media was entirely removed from the discs and kept at 0° C., while fresh media was added to continue the elution over a total period of time of 10 days. The DMEM solution containing eluted drug was used to perform the assay. Results demonstrate that anti-proliferative therapeutic compounds can be retained onto stainless steel 316L discs for sustained release to effectively inhibit HSV-SMC proliferation for a period of time of up to 10 days with either actinomycin D, rapamycin, paclitaxel, doxorubicin, and colchicine. Bromobenzenediazonium alone does not inhibit cell proliferation therefore, demonstrating that the observed anti-proliferative effect is not caused by potential elution of the organic layer (composed of the electrodeposited bromobenzenediazonium molecule). When actinomycin D was deposited on uncoated discs, the drug was rapidly eluted from the discs, preventing HSV-SMC proliferation for up to 24 hours. After 24 hours, it is apparent from FIG. 10A that little drug is retained on bare stainless steel discs, emphasizing the necessity of the coating with the diazonium-containing molecule for sustained release of drugs. Rapamycin, colchicine, and paclitaxel were also retained onto the disc for slow elution. Doxorubicin is a potent anti-proliferative drug, which is hydrophilic in nature. Therefore, the bulk of the drug is released within the first 24 hours, leaving little drug onto the disc for subsequent inhibition of proliferation at later time points, thus proving the necessity of the lipophilic nature of the drug.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

What is claimed is:

1. A method for loading a drug onto an endovascular device, said method comprising the steps of :
electrodepositing an hydrophobic molecule containing a diazonium moiety onto the surface of an endovascular device to obtain a functionalized surface of said device; and
depositing passively a lipophilic drug onto said functionalized surface, said drug binding to the diazonium moiety of the molecule for slow elution into a tissue when said device is brought in contact with said tissue in vivo.

2. The method of claim 1, wherein the endovascular device is made of stainless steel.

3. The method of claim 2, wherein the hydrophobic molecule is selected from the group consisting of 4-decycloxyphenyl diazonium chloride zinc chloride, 3-ethoxycarbonyl naphtalene diazonium tetrafluoroborate, 3, 5-dichlorophenyl diazonium tetrafluoroborate, 2-chloro-4-benzamido-5-methylbenzene diazonium chloride hemizinc chloride, and 4-bromobenzene diazonium tetrafluoroborate.

4. The method of claim 2, wherein the drug is selected from the group consisting of anti-proliferative agent, anti-inflammatory agent, anti-thrombotic drug, bioactive agent which promotes healing of a tissue, anti-neoplastic drug, anti-coagulant, fibrinolytic agent, non-steroidal anti-inflammatory drug (NSAID), steroidal anti-inflammatory drug, sodium channel blocker and calcium channel blocker, nitric oxide donor, alpha-adrenoceptor blocker, genetic material containing DNA and RNA, antibody, prostaglandin, leukotriene, elastin, collagen, integrin, growth factor, radioactive molecule.

5. The method of claim 4, wherein the anti-neoplastic drug is selected from the group consisting of alkylating agent, antimetabolite, antibiotic, mitotic inhibitor, hormone.

6. The method of claim 5, wherein the alkylating agent is cisplatin or melphalan.

7. The method of claim 5, wherein the antimetabolite is methotraxate or 5-fluorouracil.

8. The method of claim 5, wherein the antibiotic is actinomycin D, bleomycin or rapamycin.

9. The method of claim 5, wherein the mitotic inhibitor is selected from the group consisting of vincristine, vinblastine, paclitaxel, and colchicine.

10. The method of claim 5, wherein the hormone is prednisone or tamoxifen.

11. The method of claim 4, wherein the fibrinolytic agent is streptokinase or urokinase.

12. The method of claim 4, wherein the NSAID is ibuprofen or naproxen.

13. The method of claim 4, wherein the steroidal anti-inflammatory drug is prednisone.

14. The method of claim 4, wherein the sodium channel blocker is lidocaine or procainamide.

15. The method of claim 4, wherein the calcium channel blocker is nifedipine or verapamil.

16. The method of claim 4, wherein the nitric oxide donor is nitroglycerin.

17. The method of claim 4, wherein the alpha-adrenoceptor blocker is phentolamine or prazosin.

18. The method of claim 4, wherein the anti-coagulant is heparin or coumarin.

19. The method of any one of claims 1 to 18, wherein the step of depositing passively the drug is effected in an organic solvent.

20. The method of claim 19, wherein the organic solvent is ethanol or acetonitrile.

21. A drug-eluting endovascular device comprising:
an endovascular device;
an hydrophobic linker molecule containing a diazonium moiety electrodeposited onto the surface of the endovascular device; and
a lipophilic drug passively deposited on the linker molecule, said drug binding to the linker molecule through hydrophobic interactions for elution from the endovascular device over time.

22. The endovascular device of claim 21, wherein the device is selected from the group consisting of balloon-expandable stent, self-expandable stent, and graft.

23. The endovascular device of claim 21, wherein said endovascular device is made of stainless steel.

24. The endovascular device of claim 23, wherein the hydrophobic linker molecule is selected from the group consisting of 4-decycloxyphenyl diazonium chloride zinc chloride, 3-ethoxycarbonyl naphtalene diazonium tetrafluoroborate, 3,5-dichlorophenyl diazonium tetrafluoroborate, 2-chloro-4-benzamido-5-methylbenzene diazonium chloride hemizinc chloride, and 4-bromobenzene diazonium tetrafluoroborate.

25. The endovascular device of claim 23, wherein the drug is selected from the group consisting of anti-proliferative agent, anti-inflammatory agent, anti-thrombotic drug, conversion enzyme inhibitor, bioactive agent which promotes healing of a tissue, anti-neoplastic drug, anti-coagulant, fibrinolytic agent, non-steroidal anti-inflammatory drug (NSAID), steroidal anti-inflammatory drug, sodium channel blocker and calcium channel blocker, nitric oxide donor, alpha-adrenoceptor blocker, genetic material containing DNA and RNA, antibody, prostaglandin, leukotriene, elastin, collagen, integrin, growth factor, radioactive molecule.

26. The endovascular device of claim 25, wherein the anti-neoplastic drug is selected from the group consisting of alkylating agent, antimetabolite, antibiotic, mitotic inhibitor, hormone.

27. The endovascular device of claim 26, wherein the alkylating agent is cisplatin or melphalan.

28. The endovascular device of claim 26, wherein the antimetabolite is methotraxate or 5-fluorouracil.

29. The endovascular device of claim 26, wherein the antibiotic is actinomycin D, bleomycin or rapamycin.

30. The endovascular device of claim 26, wherein the mitotic inhibitor is selected from the group consisting of vincristine, vinblastine, paclitaxel, and colchicine.

31. The endovascular device of claim 26, wherein the hormone is prednisone or tamoxifen.

32. The endovascular device of claim 25, wherein the fibrinolytic agent is streptokinase or urokinase.

33. The endovascular device of claim 25, wherein the NSAID is ibuprofen or naproxen.

34. The endovascular device of claim 25, wherein the steroidal anti-inflammatory drug is prednisone.

35. The endovascular device of claim 25, wherein the sodium channel blocker is lidocaine or procainamide.

36. The endovascular device of claim 25, wherein the calcium channel blocker is nifedipine or verapamil.

37. The endovascular device of claim 25, wherein the nitric oxide donor is nitroglycerin.

38. The endovascular device of claim 25, wherein the alpha-adrenoceptor blocker is phentolamine or prazosin.

39. The endovascular device of claim 25, wherein the anti-coagulant is heparin or coumarin.

40. The endovascular device of claim 23 characterized in that said device is a stent or a coil.
